(19) United States
(12) Patent Application Publication
(10) Pub. No.: US 2011/0184014 A1
(43) Pub. Date: Jul. 28, 2011

(54) NEW COMPOUNDS

(76) Inventors: Katalin Nogradi, Budapest (HU); Gyorgy Keseru, Berkenye (HU); Attila Bielik, Orkeny (HU); Tamas Gati, Budapest (HU); Krisztina Gal, Budapest (HU); Monika Vastag, Budapest (HU); Amrita Agnes Bobok, Budapest (HU)

(21) Appl. No.: 12/158,589

(22) PCT Filed: Dec. 19, 2006
(86) PCT No.: PCT/HU2006/000119

§ 371 Date: Dec. 22, 2008

(30) Foreign Application Priority Data
Dec. 20, 2005 (HU) P0501170

(51) Int. Cl. A61K 31/4365; C07D 495/04; A61P 25/00

(52) U.S. Cl. 514/301; 546/114

(57) ABSTRACT

The present invention relates to new mGluR1 and mGluR5 receptor subtype preferring ligands of formula (I); wherein Y represents a substituent selected from hydrogen, methyl, fluoro, chloro, bromo, methoxy; Z is hydrogen or methyl; R is an optionally substituted heteroaryl, and/or salts and/or hydrates and/or solvates thereof, to the processes for producing the same, to pharmaceutical compositions containing the same and to their use in therapy and/or prevention of pathological conditions which require the modulation of mGluR1 and mGluR5 receptors such as neurological disorders, psychiatric disorders, acute and chronic pain and neuromuscular dysfunctions of the lower urinary tract.

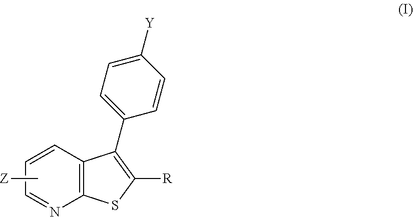

### FIELD OF THE INVENTION

The present invention relates to new mGluR1 and mGluR5 receptor subtype preferring ligands of formula (I) and/or salts and/or hydrates and/or solvates thereof, to the processes for their preparation, to pharmaceutical compositions containing these compounds and to their use in therapy and/or prevention of a condition which requires modulation of mGluR1 and mGluR5 receptors.

### BACKGROUND OF THE INVENTION

A major excitatory neurotransmitter in the mammalian central nervous system (CNS) is the glutamate molecule, which binds to neurons, thereby activating cell surface receptors. These receptors can be divided into two major classes, ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A2; increases in arachidonic acid release; and increases or decreases in the activity of voltage- and ligand-gated ion channels. (Trends Pharmacol. Sci., 1993, 14, 13; Neurochem. Int., 1994, 24, 439; Neuropharmacology, 1995, 34, 1; Prog. Neurobiol., 1999, 59, 55; Berl. Psychopharmacology 2005, 179, 4).

Eight distinct mGluR subtypes, termed mGluR1 through mGluR8, have been identified by molecular cloning (Neuron, 1994, 13, 1031; Neuropharmacology, 1995, 34, 1; J. Med. Chem., 1995, 38, 1417). Further receptor diversity occurs via expression of alternatively spliced forms of certain mGluR subtypes (PNAS, 1992, 89, 10331; BBRC, 1994, 199, 1136; J. Neurosci., 1995, 15, 3970).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants.

Attempts at elucidating the physiological roles of Group I mGluRs suggest that activation of these receptors elicits neuronal excitation. Evidence indicates that this excitation is due to direct activation of postsynaptic mGluRs, but it also has been suggested that activation of presynaptic mGluRs occurs, resulting in increased neurotransmitter release (Trends Pharmacol. Sci., 1992, 15, 92; Neurochem. Int., 1994, 24, 439; Neuropharmacology, 1995, 34, 1; Trends Pharmacol. Sci., 1994, 15, 33).

Metabotropic glutamate receptors have been implicated in a number of normal processes in the mammalian CNS. Activation of mGluRs has been shown to be required for induction of hippocampal long-term potentiation and cerebellar long-term depression (Nature, 1993, 363, 347; Nature, 1994, 368, 740; Cell, 1994, 79, 365; Cell, 1994, 79, 377). A role for mGluR activation in nociception and analgesia also has been demonstrated (Neuroreport, 1993, 4, 879; Brain Res., 1999, 871, 223).

Group I metabotropic glutamate receptors, both mGluR5 and mGluR1 have been suggested to play roles in a variety of pathophysiological processes and disorders affecting the CNS. These include anxiety, depression, stroke, head trauma, anoxic and ischemic injuries, hypoglycemia, epilepsy, neurodegenerative disorders such as Alzheimer's disease, GERD and pain (Trends Pharmacol. Sci., 1993, 14, 13; Life Sci. 1994, 54, 135; Ann. Rev. Neurosci., 1994, 17, 31; Neuropharmacology, 1995, 34, 1; J. Med. Chem., 1995, 22, 331; Trends Pharmacol. Sci., 2001, 22, 331; Curr. Opin. Pharmacol., 2002, 2, 43; Pain, 2002, 98, 1; Neuropsychopharmacology 2004, 1; Pharm. Biochem. Behav., 2005, 81, 901; Gastroenterology, 2005, 128, 402; Pain, 2005, 114, 195). Further, mGluR5-selective compounds such as 2-methyl-6-(phenylethynyl)-pyridine (“MPEP”) are effective in animal models of mood disorders, including anxiety and depression (J. Pharmacol. Exp. Ther., 2000, 295, 1267; Brit. J. Pharmacol., 2001, 132, 1423; Pol. J. Pharmacol., 2001, 132, 1423). Selective mGluR1 compounds are also proved to be effective in animal models of anxiety, pain and neuroprotection (Eur. J. Pharmacol., 2004, 492, 137; Pharmacology, 2005, 179, 207; Pain, 2005, 113, 211; Ann. NY Acad. Sci., 2005, 1053, 55-73; Neuropharmacology, 2005, 49, Suppl. 1.)

Much of the pathology in these conditions is thought to be due to excessive glutamate-induced excitation of CNS neurons. As Group I mGluRs (mGluR1 and mGluR5) appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Japanese Patent JP 07076586 describes furopyridines and thienopyridines as bone absorption inhibitors for the treatment of osteoporosis.

Thienopyridine derivatives are useful as hematinics, antitumor agents and immunostimulants, as described in JP 07053562 patent application.

According to E. Zeinab et al. (Arch. Pharm, 1992, 325(5), 301) thienopyridine and thienopyrimidine derivatives were synthesized and their mycotoxin inhibitor activities were evaluated. Some of the compounds inhibit the production of mycotoxins and fungal growth.

The compounds mentioned in the above publications are not declared or even not suggested having activity on the mGluR receptors.

### SUMMARY OF THE INVENTION

The present invention relates to new mGluR1 and mGluR5 receptor subtype preferring ligands of formula (I):

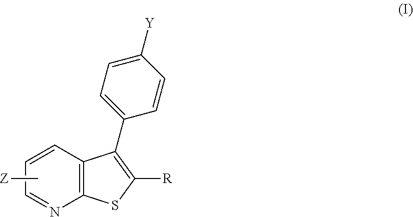

wherein

Y represents a substituent selected from hydrogen, methyl, fluoro, chloro, bromo, methoxy;

Z is hydrogen or methyl;

R is an optionally substituted heteroaryl, and/or salts and/or hydrates and/or solvates thereof, to the processes for producing the same, to pharmaceutical compositions containing the same and to their use in therapy and/or prevention of pathological conditions which require the modulation of mGluR1 and mGluR5 receptors such as neurological disorders, psychiatric disorders, acute and chronic pain and neuromuscular dysfunctions of the lower urinary tract.

### DETAILED DESCRIPTION OF THE INVENTION

The present invention relates to new mGluR1 and mGluR5 receptor subtype preferring ligands of formula (I):

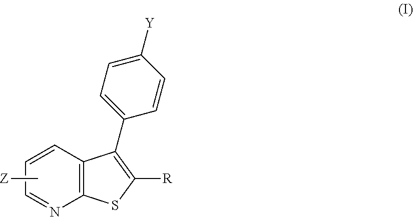

wherein

Y represents a substituent selected from hydrogen, methyl, fluoro, chloro, bromo, methoxy;

Z is hydrogen or methyl;

R is an optionally substituted heteroaryl, and/or salts and/or hydrates and/or solvates thereof.

More preferred compounds of this invention include compounds of formula (I) having the structure

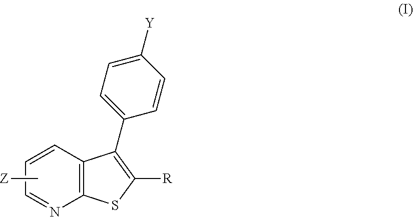

wherein

Y represents a substituent selected from hydrogen, methyl, fluoro, chloro, bromo, methoxy;

Z is hydrogen or methyl;

R is a monocyclic or bicyclic heteroaryl ring containing 1-4 heteroatom(s) selected from O, N or S, which is optionally substituted with one or more alkyl, alkoxy, halogen, methoxycarbonyl, amino, alkylamino, acylamino, optionally substituted phenyl or a monocyclic or bicyclic heteroaryl ring containing 1-4 heteroatom(s) selected from O, N or S;

and/or salts and/or hydrates and/or solvates thereof.

The heteroaryl group may be a monocyclic or bicyclic aromatic ring containing 1-4 heteroatom(s) selected from O, N or S such as thiazolyl, oxazolyl, isoxazolyl, oxadiazolyl, furyl etc. ring.

The heteroaryl group may be optionally substituted with one or more methyl, methoxy, fluoro, chloro, bromo, methoxycarbonyl, amino, alkylamino, acylamino, monocyclic or bicyclic aromatic ring containing 1-4 heteroatom(s) selected from O, N or S, such as pyridyl, thiophen ring or phenyl optionally substituted with one or more halogen group.

Compounds of formula (I) may form salts with acids. The invention relates also to the salts of compounds of formula (I) formed with acids, especially the salts formed with pharmaceutically acceptable acids. The meaning of compound of formula (I) is either the free base or the salt even if it is not referred separately.

Both organic and inorganic acids can be used for the formation of acid addition salts. Suitable inorganic acids can be for example hydrochloric acid, sulfuric acid, nitric acid and phosphoric acid. Representatives of monovalent organic acids can be for example formic acid, acetic acid, propionic acid, and different butyric acids, valeric acids and capric acids. Representatives of bivalent organic acids can be for example oxalic acid, malonic acid, maleic acid, fumaric acid and succinic acid. Other organic acids can also be used, such as hydroxy acids for example citric acid, tartaric acid, or aromatic carboxylic acids for example benzoic acid or salicylic acid, as well as aliphatic and aromatic sulfonic acids for example methanesulfonic acid, naphthalenesulfonic acid and p-toluenesulfonic acid. Especially valuable group of the acid addition salts is in which the acid component itself is physiologically acceptable and does not have therapeutical effect in the applied dose or it does not have unfavourable influence on the effect of the active ingredient. These acid addition salts are pharmaceutically acceptable acid addition salts. The reason why acid addition salts, which do not belong to the pharmaceutically acceptable acid addition salts belong to the present invention is, that in given case they can be advantageous in the purification and isolation of the desired compounds.

Solvates and/or hydrates of compounds of formula (I) are also included within the scope of the invention.

Especially important compounds of formula (I) of the present invention are the following:
3-(4-fluoro-phenyl)-2-(5-methyl-isoxazol-3-yl)-thieno[2,3-b]pyridine,
3-(4-chloro-phenyl)-2-(2-pyridin-2-yl-thiazol-4-yl)-thieno[2,3-b]pyridine,
3-(4-chloro-phenyl)-2-(2-thiophen-2-yl-oxazol-4-yl)-thieno[2,3-b]pyridine,
{4-[3-(4-chloro-phenyl)-thieno[2,3-b]pyridin-2-yl]-thiazol-2-yl}-ethyl-amine,
N-{4-[3-(4-chloro-phenyl)-thieno[2,3-b]pyridin-2-yl]-thiazol-2-yl}-acetamide,
3-(4-chloro-phenyl)-6-methyl-2-(5-methyl-isoxazol-3-yl)-thieno[2,3-b]pyridine,
3-(4-chloro-phenyl)-2-(5-methyl-isoxazol-3-yl)-thieno[2,3-b]pyridine,
5-[3-(4-chloro-phenyl)-thieno[2,3-b]pyridin-2-yl]-2-methyl-furan-3-carboxylic acid methyl ester,
3-(4-chloro-phenyl)-2-(3-ethyl-[1,2,4]oxadiazol-5-yl)-thieno[2,3-b]pyridine,
3-(4-chloro-phenyl)-2-[3-(4-fluoro-phenyl)-[1,2,4]oxadiazol-5-yl]-thieno[2,3-b]pyridine,
3-(4-fluoro-phenyl)-2-[5-(4-fluoro-phenyl)-4,5-dihydro-isooxazol-3-yl]-thieno[2,3-b]pyridine.

Pharmaceutical Formulations

The invention also relates to the pharmaceutical compositions containing the compounds of formula (I) and/or physiologically acceptable salts and/or hydrates and/or solvates thereof as active ingredient and one or more physiologically acceptable carriers.

The compounds of formula (I) and/or physiologically acceptable salts and/or hydrates and/or solvates thereof may be administered by any convenient method, for example by oral, parenteral (including subcutaneous, intramuscular, and intravenous), buccal, sublingual, nasal, rectal or transdermal administration and the pharmaceutical compositions adapted accordingly.

The compounds of formula (I) and/or physiologically acceptable salts and/or hydrates and/or solvates thereof which are active when given orally can be formulated as liquids or solids, for example syrups, suspensions or emulsions, tablets, capsules and lozenges.

A liquid formulation of the compounds of formula (I) and/or physiologically acceptable salts and/or hydrates and/or solvates thereof generally consist of a suspension or solution of the compound of formula (I) and/or physiologically acceptable salts and/or hydrates and/or solvates thereof in a suitable liquid carrier(s) for example an aqueous solvent, such as water and ethanol or glycerine, or a non-aqueous solvent, such as polyethylene glycol or an oil. The formulation may also contain a suspending agent, preservative, flavouring or colouring agent.

A composition in the solid form of a tablet can be prepared using any suitable pharmaceutical carrier(s) routinely used for preparing solid formulations. Examples of solid carriers include lactose, terra alba, sucrose, talc, gelatine, agar, pectin, acacia, magnesium stearate, stearic acid etc. Optionally, tablets may be coated by standard aqueous or nonaqueous techniques.

A composition in the solid form of a capsule can be prepared using routine encapsulation procedures. For example, pellets containing the active ingredient can be prepared using standard carriers and then these are filled into a hard gelatine capsule; alternatively, a dispersion or suspension can be prepared using any suitable pharmaceutical carrier(s), for example aqueous gums, celluloses, silicates or oils and the dispersion or suspension then is filled into a soft gelatine capsule.

Typical parenteral compositions consist of a solution or suspension of the compound of formula (I) and/or physiologically acceptable salts and/or hydrates and/or solvates thereof in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions of the present invention for nasal administration containing a compound of formula (I) and/or physiologically acceptable salts and/or hydrates and/or solvates thereof may conveniently be formulated as aerosols, drops, gels and powders. Aerosol formulations of the present invention typically comprise a solution or fine suspension of the compound of formula (I) and/or physiologically acceptable salts and/or hydrates and/or solvates in a physiologically acceptable aqueous or non-aqueous solvent and are usually presented in a single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal once the contents of the container have been exhausted. If the dosage form comprises an aerosol dispenser, it will contain a propellant which can be a compressed gas, such as compressed air or an organic propellant, such as a fluorochlorohydrocarbon. The aerosol dosages form can also take the form of a pump-atomiser.

Compositions of the present invention containing a compound of formula (I) and/or physiologically acceptable salts and/or hydrates and/or solvates are suitable for buccal or sublingual administration including tablets, lozenges and pastilles, wherein the active ingredient is formulated with a carrier, such as sugar and acacia, tragacanth, or gelatine, glycerin etc.

Compositions of the present invention containing a compound of formula (I) and/or physiologically acceptable salts and/or hydrates and/or solvates thereof for rectal administration are conveniently in the form of suppositories containing a conventional suppository base, such as cocoa butter and other materials commonly used in the art. The suppositories may be conveniently formed by first admixing the composition with the softened or melted carrier(s) followed by chilling and shaping in moulds.

Compositions of the present invention containing a compound of formula (I) and/or physiologically acceptable salts and/or hydrates and/or solvates thereof for transdermal administration include ointments, gels and patches.

The compositions of the present invention containing a compound of formula (I) and/or physiologically acceptable salts and/or hydrates and/or solvates thereof is preferably in the unit dose form, such as tablet, capsule or ampoule.

Each dosage unit of the present invention for oral administration contains preferably from 0.1 to 500 mg of a compound of formula (I) and/or physiologically acceptable salts and/or hydrates and/or solvates thereof calculated as a free base.

Each dosage unit of the present invention for parenteral administration contains preferably from 0.1 to 500 mg of a compound of formula (I) and/or physiologically acceptable salts and/or hydrates and/or solvates thereof calculated as a free base.

The compounds of formula (I) and/or physiologically acceptable salts and/or hydrates and/or solvates thereof can normally be administered in a daily dosage regimen. In the treatment of mGluR1 and mGluR5 mediated disorders, such as schizophrenia, anxiety, depression, panic, bipolar disorders, and circadian disorders or chronic and acute pain disorders the dosage levels from about 0.01 mg/kg to about 140 mg/kg of body weight per day are useful or alternatively about 0.5 mg to about 7 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 250-300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

Medical Use

The compounds of formula (I) of the present invention have been found to exhibit biological activity at mGluR1 and mGluR5 receptors and are expected to be useful in the treatment of mGluR1 and mGluR5 mediated disorders.

It has been found that the compounds according to the present invention or salts thereof, exhibit a high degree of potency and selectivity for individual metabotropic glutamate receptor (mGluR) subtypes. In particular there are compounds according to the present invention that are potent and selective for mGluR1 and mGluR5 receptors. Accordingly, the compounds of the present invention are expected to be useful in the prevention and/or treatment of conditions associated with excitatory activation of mGluR1 and mGluR5 receptor and for inhibiting neuronal damage caused by excitatory activation of mGluR1 and mGluR5 receptor. The compounds may be used to produce an inhibitory effect of mGluR1 and mGluR5, in mammals, including human.

Thus, it is expected that the compounds of the invention are well suited for the prevention and/or treatment of mGluR1 and mGluR5 receptor-mediated disorders such as acute and chronic neurological and psychiatric disorders, chronic and acute pain disorders and neuromuscular dysfunctions of the lower urinary tract.

The dose required for the therapeutic or preventive treatment of a particular disorder will necessarily be varied depending on the host treated and the route of administration.

The invention relates to compounds of formula (I) as defined hereinbefore, for use in therapy.

The invention relates to compounds of formula (I) as defined hereinbefore, for use in prevention and/or treatment of mGluR1 and mGluR5 receptor-mediated disorders.

The invention relates to compounds of formula (I) as defined hereinbefore, for use in prevention and/or treatment of neurological disorders.

The invention relates to compounds of formula (I) as defined hereinbefore, for use in prevention and/or treatment of psychiatric disorders.

The invention relates to compounds of formula (I) as defined hereinbefore, for use in prevention and/or treatment of chronic and acute pain disorders.

The invention relates to compounds of formula (I) as defined hereinbefore, for use in prevention and/or treatment of neuromuscular dysfunctions of the lower urinary tract.

The invention relates to compounds of formula (I) as defined hereinbefore, for use in prevention and/or treatment of pain related to migraine, inflammatory pain, neuropathic pain disorders such as diabetic neuropathies, arthritis and rheumatoid diseases, low back pain, post-operative pain and pain associated with various conditions including angina, in renal or biliary colic, menstruation, migraine and gout.

The invention relates to compounds of formula (I) as defined hereinbefore, for use in prevention and/or treatment of Alzheimer's disease senile dementia, AIDS-induced dementia Parkinson's disease, amyotrophic lateral sclerosis, Huntington's Chorea, migraine, epilepsy, schizophrenia, depression, anxiety, acute anxiety, obsessive compulsive disorder, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula (I) as defined hereinbefore, for use in prevention and/or treatment of stroke, head trauma, anoxic and ischemic injuries, hypoglycemia, cardiovascular diseases and epilepsy.

The compounds are also well suited for the treatment of neuromuscular dysfunction of the lower urinary tract, such as urinary urgency, overactive bladder, greater urinary frequency, reduced urinary compliance, cystitis, incontinence, enuresis and dysuria.

The present invention relates also to the use of a compound of formula (I) as defined hereinbefore, in the manufacture of a medicament for the prevention and/or treatment of mGluR1 and mGluR5 receptor-mediated disorders and any disorder listed above.

The invention also provides a method of treatment and/or prevention of mGluR1 and mGluR5 receptor mediated disorders and any disorder listed above, in a patient suffering from, or at risk of, said condition, which comprises administering to the patient an effective amount of a compound of formula (I), as hereinbefore defined.

In the context of the present specification, the term “therapy” includes treatment as well as prevention, unless there are specific indications to the contrary. The terms “therapeutic” and “therapeutically” should be construed accordingly.

In this specification, unless stated otherwise, the term “antagonist” means a compound that by any means, partly or completely blocks the transduction pathway leading to the production of a response by the ligand.

The term “disorder”, unless stated otherwise, means any condition and disease associated with metabotropic glutamate receptor activity.

Methods of Preparation

Abbreviation

The abbreviation used herein have the following tabulated meaning. Abbreviations not tabulated below have their meanings as commonly used unless specifically stated otherwise.

DMF N,N-dimethylformamide
According to the present invention a process for the preparation of a compound of formula (I)

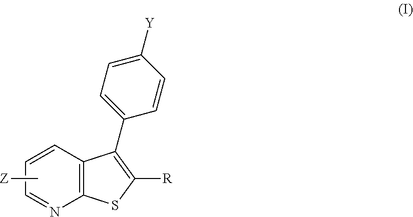

wherein Y represents a substituent selected from hydrogen, methyl, fluoro, chloro, bromo, methoxy;

Z is hydrogen or methyl;

R is an optionally substituted heteroaryl,

and/or salts and/or hydrates and/or solvates thereof

by reacting a compound of formula (IV):

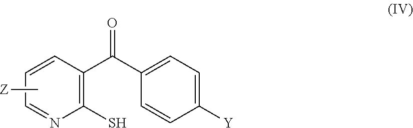

wherein the meaning of Z and Y is as described above for the formula (I),

with a compound of formula (VI):

HlgCH₂R (VI)

wherein Hlg is chloro or bromo, R is as defined in claims 1 and 2,
in the presence of a base in a solvent under reflux or in a microwave reactor,
and optionally thereafter forming salts and/or hydrates and/or solvates of compounds of formula (I).

Compounds of the present invention can be prepared according to the following method. Unless stated otherwise, the meaning of substituents is as defined above for formula I or apparent to one skilled in the art.

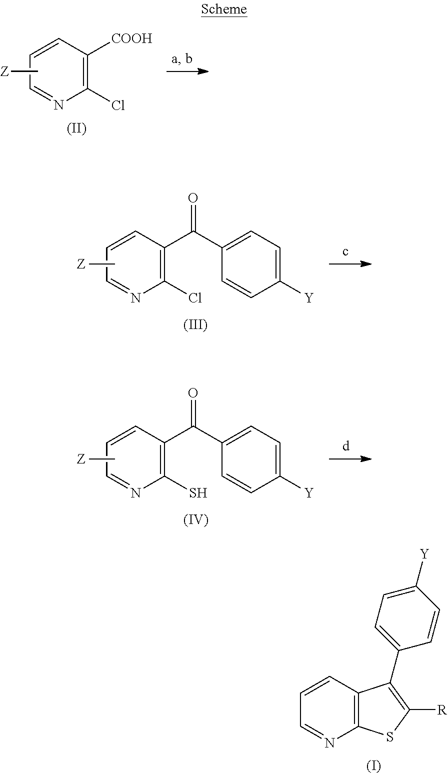

a. SOCl₂, benzene substituted with Y (Ph-Y, compounds of formula (V)), catalytic amount of DMF, 80-130° C., 2-3 hours;

b. AlCl₃, 80-130° C., 5-8 hours;

c. thiourea, water/ethanol, reflux, 20-24 hours;

d. Halomethyl heterocycles (HlgCH₂R compounds of formula (VI), wherein Hlg means chloro or bromo, R is a heteroaryl group, which can be a monocyclic or bicyclic ring containing 1-4 heteroatom(s) selected from O, N or S, and which is optionally substituted with one or more alkyl, alkoxy, halogen, methoxycarbonyl, amino, alkylamino, acylamino, optionally substituted phenyl or a heteroaryl group, which is a monocyclic or bicyclic ring containing 1-4 heteroatom(s) selected from O, N or S; base e.g. NaOCH₃ or KOtBu, solvent e.g. methanol, ethanol or DMF, 60-150° C., 2-24 hours; or in some cases halomethyl heterocycles, Cs₂CO₃, DMF, microwave, 200° C., 20-60 minutes).

Acid chloride was prepared from the appropriate 2-chloro-nicotinic acid derivative by the reaction of thionylchloride with the benzene or with the appropriate benzene derivative in the presence of AlCl₃. The reaction may be carried out by well-known methods suitable for Friedel-Crafts reactions using benzene or the appropriate benzene derivative as solvent.

The product (III) was purified by crystallization and reacted with thiourea in a mixture of water and ethanol under reflux according to the method of J. Katritzky (J. Chem. Soc., 1958, 3610). The resulted compounds of formula (IV) are in crystalline form.

Compounds of formula (IV) were reacted with different optionally substituted halomethyl-heterocyclic derivatives in the presence of a base (e.g. NaOMe, KOtBu or Cs₂CO₃). The halomethyl compounds are either commercially available building blocks from e.g. Aldrich and Enamine Ltd, or can be prepared analogous to known methods. The reaction was carried out in the appropriate solvent (e.g. methanol, ethanol or dimethylformamide) between 60-150° C.

In some cases the preparation of compounds of formula (I) (e.g. when halomethyl-heterocyclic compound was 5-chloromethyl-2-methyl-furan-3-carboxylic acid methylester) the reaction was carried out in a microwave apparatus (detailed description of the apparatus see later) at 200° C. applied 18 bar and 300 Watt during 20-60 minutes.

The obtained compounds of formula (I) was purified by crystallization or by column chromatography.

Compounds of formula (I) can be transformed into the salts thereof with acids and/or can be liberated from the obtained acid addition salts by treatment with a base.

Compounds of formula (I) can be transformed into hydrates and/or solvates.

Biological Test Methods

MGluR1 Receptor Binding Test

MGluR1 receptor binding testes were performed according to modified method of Lavreysen et al. (Mol. Pharm., 2003, 63, 1082). Based on the high homology between the human and rat mGluR1 receptors, rat cerebellar membrane preparation was used to determine the binding characteristics of reference compounds and novel compounds to the rat mGluR1. As radioligand [3H]R214127 (3 nM) was used and the nonspecific binding was determined in the presence of 1 μM of R214127.

IC-50 values were determined from displacement curves by nonlinear regression analysis and were converted by equation method of Cheng and Prusoff (Biochem. Pharmacol., 1973, 22, 3099) to Kᵢ values.

MGluR5 Receptor Binding Tests

MGluR5 receptor binding was determined according to Gasparini et. al. (Bioorg. Med. Chem. Lett. 2000, 12:407-409) with modifications. Rat cerebro-cortical membrane preparation was used to determine the binding characteristics of reference compounds and novel compounds to the rat mGluR5. The A18 cell line expressing hmGluR5a (purchased from Euroscreen) was used to determine binding characteristics of the chemical compounds to the human mGluR5a receptor. As radioligand [3H]-M-MPEP (2 nM) was used. The nonspecific binding was determined in the presence of 10 μM M-MPEP,

### Assessment of Functional Activity

Cell Cultures for Native Rat mGluR5 and mGluR1 Receptors

Functional potency at native rat mGluR5 and mGluR1 receptors was estimated using primary neocortical cell cultures derived from 17 day old Charles River rat embryos and primary cerebellar cell cultures derived from 4-day old Wistar rats, respectively (for the details on the preparation of neural cell cultures see Johnson, M. I.; Bunge, R. P. (1992): Primary cell cultures of peripheral and central neurons and glia. In: Protocols for Neural Cell Culture, eds: Fedoroff, S.; Richardson A., The Humana Press Inc., 51-77). After isolation the cells were plated onto standard 96-well microplates and the cultures were maintained in an atmosphere of 95% air-5% CO₂ at 37° C. The neocortical and cerebellar cultures were used for the calcium measurements after 5-7 and 3-4 days in vitro, respectively.

Cell Cultures for Recombinant Human mGluR5a Receptors

Chinese hamster ovary (CHO) cells stably expressing recombinant human mGluR5a (CHO-mGluR5a, purchased from Euroscreen) receptors were cultured in F12 medium containing 10% FCS, 1% antibiotic antimycotic solution, 400 μg/ml G418, 250 μg/ml zeocin, 5 μg,/ml puromycin. Cells were kept at 37° C. in a humidified incubator in an atmosphere of 5% CO₂/95% air and were passaged three times a week. Cells were plated at 2.5−3.5×104 cell/well on standard 96-well microplates, receptor expression was induced by adding 600 ng/ml doxycycline on the next day. The calcium measurements were carried out 16-24 hours after the addition of the inducing agent.

Fluorimetric Measurement of Cytosolic Calcium Concentration

Measurements of cytosolic calcium concentration ([Ca²⁺]ᵢ) were carried out on primary neocortical and cerebellar cultures, and on CHO-mGluR5a cells stably expressing human mGluR5a receptors. Cells were grown in standard 96-well microplates and before the measurement were loaded with a fluorescent Ca²⁺-sensitive dye, fluo-4/AM (2 μM): the neural cultures were loaded in their growth medium, CHO-mGluR5a cells were loaded in assay buffer (145 mM NaCl, 5 mM KCl, 2 mM MgCl₂, 2 mM CaCl₂, 10 mM HEPES, 20 mM D-glucose, 2 mM probenecid, pH=7.4) supplemented with 2 mM Na-pyruvate and 30 μg/ml glutamate-pyruvate transaminase (in case of CHO-mGluR5a cells these supplements were also present during the course of the [Ca²⁺]ᵢ measurements). Loading was done by incubating the cells with 100 μl/well dye solution at 37° C. in a humidified incubator in an atmosphere of 5% CO2/95% air for 40-120 min. To stop dye loading cells were washed twice with assay buffer. After washing, various concentrations of the test compounds (diluted in assay buffer from a DMSO or a dimethylformamide (DMF) stock solution, final DMSO/DMF concentration was <0.1%) or buffer were added to each well depending on the experimental setup. In the case of neocortical cultures the assay buffer also contained TTX (0.5 to suppress spontaneous oscillations of [Ca2+]i, in the case of cerebellar cultures probenecid was substituted with sulfinpyrazone (0.25 mM).

After incubation at 37° C. for 10-20 min. baseline and agonist-evoked changes of [Ca2+]i were measured column by column with a plate reader fluorimeter (FlexStation II, Molecular Devices). Excitation and detection of emission was carried out from the bottom of the plate. The whole measurement process was performed at 37° C. and was controlled by custom software Inhibitory potency of the test compounds was assessed by measuring the reduction in the agonist-evoked [Ca²⁺]ᵢ-elevation in the presence of different concentrations of the compounds. DHPG was used as agonist for all three cultures, the concentration was 20 and 100 μM for neocortical and cerebellar cultures, respectively. In the case of CHO-mGluR5a cells DHPG was applied at an EC80 concentration, the EC80-values were derived from daily determined dose-response curves. Fluorescence data were expressed as ΔF/F (fluorescence change normalized to baseline).

All treatments on a single plate were measured in multiple wells. Data from all wells with the same treatment were averaged and the average values were used for analysis. Inhibitory potency of a compound at a single concentration point was expressed as percent inhibition of the control agonist response. Sigmoidal concentration-inhibition curves were fitted to the data (derived from at least three independent experiments) and IC50-values were determined as the concentration that produces half of the maximal inhibition caused by the compound. Raw fluorescence data were analyzed using Soft Max Pro (Molecular Devices), curve fitting was done with GraphPad Prism.

Results

Compounds of formula (I) of the present invention showed affinity for both rat and human mGluR1 and mGluR5 receptors and proved to be functional antagonists that are they inhibited functional responses elicited by stimulation of mGluR5 receptors.

TABLE

| | | (M)⁺ | mGlu5 | mGlu1 | |
|---|---|---|---|---|---|
| Comp. | | or | Kᵢ | Kᵢ | |
| No. | Structure | (M + H)+ | (nM) | (nM) | ¹H NMR data |
| 1 | 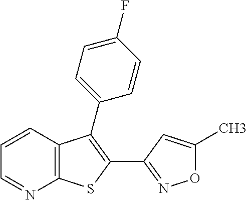 | 310 | * | * | (500 MHz, CDCl₃, 30° C.): 8.63 (dd, J = 4.6, 1.6 Hz, 1H); 7.73 (dd, J = 8.1, 1.6 Hz, 1H); 7.40-7.33 (m, 2H); 7.29 (dd, J = 8.1, 4.6 Hz, 1H); 7.25-7.19 (m, 2H); 5.44 (q, J = 0.8 Hz, 1H); 2.35 (d, J = 0.8 Hz, 3H). |
| 2 | 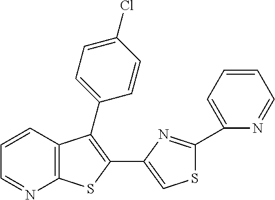 | 406 | ** | ** | (300 MHz, DMSO-d₆, 30° C.): 8.68-8.59 (m, 2H); 8.11 (dt, J = 7.8, 1.2 Hz, 1H); 8.01 (ddd, J = 7.8, 7.5, 1.7 Hz, 1H); 7.75 (dd, J = 8.2, 1.6 Hz, 1H); 7.70-7.63 (m, 2H); 7.58-7.50 (m, 3H); 7.45 (dd, J = 8.2, 4.6 Hz, 1H); 7.21 (s, 1H). |
| 3 | 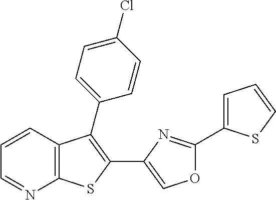 | 395 | ** | ** | (500 MHz, DMSO-d₆, 30° C.): 8.58 (dd, J = 4.6, 1.6 Hz, 1H); 7.76 (dd, J = 5.0, 1.2 Hz, 1H); 7.74 (dd, J = 8.2, 1.6 Hz, 1H); 7.70 (dd, J = 3.7, 1.2 Hz, 1H); 7.64-7.58 (m, 2H); 7.52-7.46 (m, 2H); 7.45 (s, 1H); 7.40 (dd, J = 8.2, 4.6 Hz, 1H); 7.20 (dd, J = 5.0, 3.7 Hz, 1H). |
| 4 | 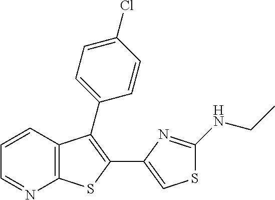 | | ** | ** | (300 MHz, DMSO-d₆, 30° C.): 8.54 (dd, J = 4.6, 1.6 Hz, 1H); 7.79 (t, J = 5.3 Hz, 1H); 7.66-7.58 (m, 3H); 7.49-7.42 (m, 2H); 7.37 (dd, J = 8.1, 4.6 Hz, 1H); 6.05 (s, 1H); 3.20 (qd, J = 7.2, 5.3 Hz, 2H); 1.14 (t, J = Hz, 3H). |
| 5 | 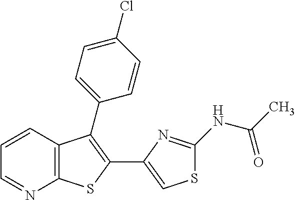 | 386 | ** | | (500 MHz, DMSO-d₆, 30° C.): 12.36 (s, 1H); 8.58 (dd, J = 4.5, 1.5 Hz, 1H); 7.69 (dd, J = 8.1, 1.5 Hz, 1H); 7.67-7.61 (m, 2H); 7.52-7.45 (m, 2H); 7.41 (dd, J = 8.1, 4.5 Hz, 1H); 6.53 (s, 1H); 2.16 (s, 3H). |
| 6 | 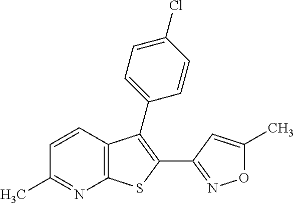 | 340 | ** | ** | (300 MHz, DMSO-d₆, 30° C.): 7.71 (d, J = 8.3 Hz, 1H); 7.67-7.60 (m, 2H); 7.50-7.42 (m, 2H); 7.35 (d, J = 8.3 Hz, 1H); 5.67 (q, J = 0.9 Hz, 1H); 2.62 (s, 3H); 2.36 (d, J = 0.9 Hz, 3H). |
| 7 | 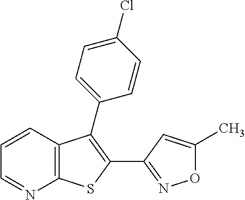 | 327 | * | * | (300 MHz, CDCl₃, 30° C.): 8.63 (dd, J = 4.6, 1.6 Hz, 1H); 7.74 (dd, J = 8.1, 1.6 Hz, 1H); 7.54-7.48 (m, 2H); 7.37-7.30 (m, 2H); 7.29 (dd, 7= 8.1, 4.6 Hz, 1H); 5.49 (q, 7 = 0.9 Hz, 1H); 2.36 (d,/ J= 0.9 Hz, 3H). |
| 8 | 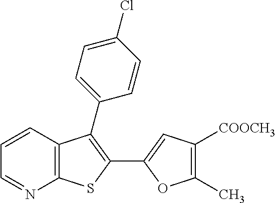 | 383 | ** | ** | (300 MHz, DMSO-d₆, 30° C.): 8.61 (dd, J = 4.6, 1.6 Hz, 1H); 7.76 (dd, J = 8.2, 1.6 Hz, 1H); 7.70-7.62 (m, 2H); 7.53-7.47 (m, 2H); 7.46 (dd, J = 8.2, 4.6 Hz, 1H); 6.28 (q, J = 0.4 Hz, 1H); 3.73 (s, 3H); 2.55 (d, J = 0.4 Hz, 3H). |
| 9 | 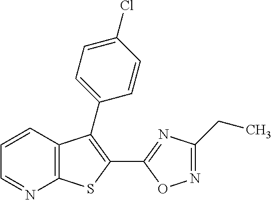 | 341 | ** | * | (300 MHz, DMSO-d₆, 30° C.): 8.75 (dd, J = 4.6, 1.6 Hz, 1H); 7.93 (dd, J = 8.2, 1.6 Hz, 1H); 7.63-7.56 (m, 2H); 7.56-7.48 (m, 3H); 2.75 (q, J = 7.6 Hz, 2H); 1.28 (t, J = 7.6 Hz, 3H). |
| 10 | 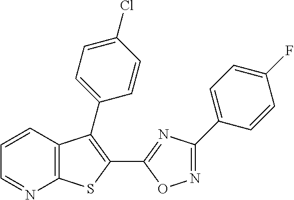 | 407 | ** | ** | (300 MHz, CDCl₃, 30° C.): 8.73 (dd, J = 4.6, 1.6 Hz, 1H); 8.12-8.03 (m, 2H); 7.88 (dd, J = 8.2, 1.6 Hz, 1H); 7.59-7.51 (m, 2H); 7.46-7.41 (m, 2H); 7.39 (dd, J = 8.2, 4.6 Hz, 1H); 7.22-7.11 (m, 2H). |
| 11 | 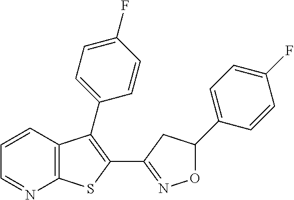 | 393 | * | * | (500 MHz, CDC1₃) 30° C.): 8.62 (dd, J = 4.6, 1.6 Hz, 1H); 7.60 (dd, J = 8.2, 1.5 Hz, 1H); 7.34-7.28 (m, 2H); 7.27-7.20 (m, 3H); 7.20-7.13 (m, 2H); 7.07-6.99 (m, 2H); 5.57 (dd, J = 10.9, 8.4 Hz, 1H); 3.11 (dd, J = 17.0, 10.9 Hz, 1H); 2.64 (dd, J = 17.0, 8.4 Hz, 1H). |

The invention is further illustrated by the following non-limiting examples.

### EXAMPLES

All starting materials are either commercially available or can be synthesized by different known methods described in the literature.

### Example 1

### (4-Chloro-phenyl)-(2-chloro-pyridin-3-yl)-methanone

Thionyl chloride (15 ml, 0.2 mol) and DMF (0.5 ml) were added dropwise to the suspension of 2-chloro-nicotinic acid (31.5 g, 0.2 mol) in chlorobenzene (100 ml) and the reaction mixture was stirred at 120° C. for 4 hours.

Aluminium chloride (33 g, 0.25 mol) was added at 0° C. to the reaction mixture, and it was boiled for 6 hours. The reaction mixture was poured onto ice (100 ml) and ethyl acetate (100 ml) was added. The mixture was stirred for half an hour at room temperature. The pH was adjusted to 8 by aqueous sodium hydroxide solution (40%). The emulsion was filtered, the filtrate was separated and extracted by ethyl acetate (2×50 ml). The organic phase was washed with water (100 ml) dried over Na₂SO₄ and concentrated in vacuo. The crude product was crystallized from isopropanol (20 ml) to yield 19.5 g (34%) of the titled compound.
In the case of the synthesis of ketones starting from substituted 2-chloro-nicotinic acids the same method was used.

### Example 2

### (4-Chloro-phenyl)-(2-mercapto-pyridin-3-yl)-methanone hydrochloride salt

The solution of thiourea (15.6 g, 0,200 mmol) in water (50 ml) and ethanol (25 ml) was added dropwise to the suspension of (4-chloro-phenyl)-(2-chloro-pyridin-3-yl)-methanone (7.65 g, 30 mmol) in ethanol (20 ml). The reaction mixture was heated for 24 hours, then cooled and stirred at 0° C. for 2-3 hours. The precipitate was filtered off, washed with water and purified by stirring with NaOH solution (2.5 g NaOH in 60 ml water) at room temperature for one hour. The mixture was filtered, and the filtrate was adjusted to pH 1 by 6 N aqueous hydrochloric acid. The product was filtered off, washed with water to yield 6.48 g (76%) of the titled compound.

### Example 3

### 3-(4-Fluoro-phenyl)-2-(5-methyl-isoxazol-3-yl)-thieno[2,3-b]pyridine

### (Compound 1)

(4-Fluoro-phenyl)-(2-mercapto-pyridin-3-yl)-methanone hydrochloride salt (prepared by the description of Example 1) (0.48 g, 2.1 mmol), 3-(bromomethyl)-5-isoxazole (0.40 g, 2.3 mmol) and NaOMe (0.16 g, 2.4 mmol) in methanol (8 ml) were heated under reflux for 3 hours. The reaction mixture was cooled, the crystalline product was filtered off and washed with methanol. This reaction resulted in 0.25 g (39%) of the titled compound.

Compounds with the exception of compound 4 were prepared by this method, from the different commercially available halomethyl building blocks.

### Example 4

### [3-(4-Chloro-phenyl)-thieno[2,3-b]pyridin-2-yl]-2-methyl-furan-3-carboxylic acid methyl ester hydrochloride

### (Compound 4)

To the solution of (4-chloro-phenyl)-(2-mercapto-pyridin-3-yl)-methanone hydrochloride salt (0.28 g, 1.0 mmol) in DMF, (5 ml) 5-chloromethyl-2-methyl-furan-3-carboxylic acid methyl ester (0.2 g, 1.05 mmol) and cesium carbonate (0.36 g, 1.1 .mmol) were added. The reaction mixture was treated in the CEM microwave reactor (8 ml tube, 300 watt, 200° C., 18 bar, 20 minutes). After evaporation in vacuo, water (10 ml) and chloroform (3×10 ml) was added to the residue. The organic phase was dried over Na₂SO₄, filtered and concentrated in vacuo. It was purified by chromatography (Kieselgehl 60, eluent:cyclohexane acetone 7:3) to yield 190 mg (47%) product, which was treated with HCl/methanol in the solution of diisopropyl-methanol mixture and gave 100 mg of the title compound.

### Example 5

### Preparation of Pharmaceutical Compositions

### a) Tablets:

0.01-50% of active ingredient of formula (I), 15-50% of lactose, 15-50% of potato starch, 5-15% of polyvinyl pyrrolidone, 1-5% of talc, 0.01-3% of magnesium stearate, 1-3% of colloid silicon dioxide and 2-7% of ultraamylopectin were mixed, then granulated by wet granulation and pressed to tablets.

b) Dragées, filmcoated tablets:

The tablets made according to the method described above were coated by a layer consisting of entero- or gastrosolvent film, or of sugar and talc. The dragées were polished by a mixture of beeswax and carnuba wax.

### c) Capsules:

0.01-50% of active ingredient of formula (I), 1-5% of sodium lauryl sulfate, 15-50% of starch, 15-50% of lactose, 1-3% of colloid silicon dioxide and 0.01-3% of magnesium stearate were thoroughly mixed, the mixture was passed through a sieve and filled in hard gelatin capsules.

### d) Suspensions:

Ingredients: 0.01-15% of active ingredient of formula (I), 0.1-2% of sodium hydroxide, 0.1-3% of citric acid, 0.05-0.2% of nipagin (sodium methyl 4-hydroxybenzoate), 0.005-0.02% of nipasol, 0.01-0.5% of carbopol (polyacrilic acid), 0.1-5% of 96% ethanol, 0.1-1% of flavoring agent, 20-70% of sorbitol (70% aqueous solution) and 30-50% of distilled water.

To solution of nipagin and citric acid in 20 ml of distilled water, carbopol was added in small portions under vigorous stirring, and the solution was left to stand for 10-12 h. Then the sodium hydroxide in 1 ml of distilled water, the aqueous solution of sorbitol and finally the ethanolic raspberry flavor were added with stirring. To this carrier the active ingredient was added in small portions and suspended with an immersing homogenizator. Finally the suspension was filled up to the desired final volume with distilled water and the suspension syrup was passed through a colloid milling equipment.

### e) Suppositories:

For each suppository 0.01-15% of active ingredient of formula (I) and 1-20% of lactose were thoroughly mixed, then 50-95% of adeps pro suppository (for example Witepsol 4) was melted, cooled to 35° C. and the mixture of active ingredient and lactose was mixed in it with homogenizator. The obtained mixture was mould in cooled forms.

### f) Lyophilized Powder Ampoule Compositions:

A 5% solution of mannitol or lactose was made with bidistilled water for injection use, and the solution was filtered so as to have sterile solution. A 0.01-5% solution of the active ingredient of formula (I) was also made with bidistilled water for injection use, and this solution was filtered so as to have sterile solution. These two solutions were mixed under aseptic conditions, filled in 1 ml portions into ampoules, the content of the ampoules was lyophilized, and the ampoules were sealed under nitrogen. The contents of the ampoules were dissolved in sterile water or 0.9% (physiological) sterile aqueous sodium chloride solution before administration.

1-17. (canceled)

18. A compound of formula (I): 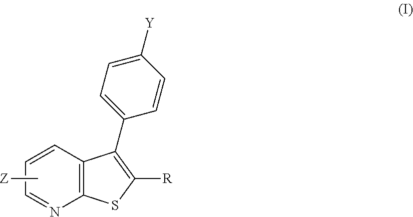
wherein:
Y is selected from hydrogen, alkyl, halogen, and alkoxy;
Z is hydrogen or alkyl; and,
R is an optionally substituted heteroaryl;
or salts or hydrates or solvates of thereof.

19. A compound of formula (I): 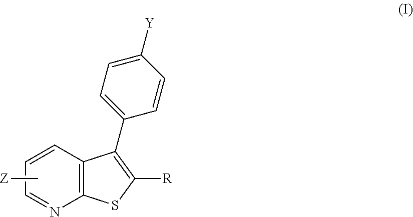
wherein:
Y is selected from hydrogen, methyl, fluoro, chloro, bromo, and methoxy;
Z is hydrogen or methyl; and,
R is a monocyclic or bicyclic heteroaryl ring containing 1-4 heteroatom(s) selected from O, N and S, which is optionally substituted with one or more alkyl, alkoxy, halogen, methoxycarbonyl, amino, alkylamino, acylamino, optionally substituted phenyl or a monocyclic or bicyclic heteroaryl ring containing 1-4 heteroatom(s) selected from O, N and S;
or salts or hydrates or solvates thereof.

20. A compound selected from:
3-(4-fluoro-phenyl)-2-(5-methyl-isoxazol-3-yl)-thieno[2,3-b]pyridine,
3-(4-chloro-phenyl)-2-(2-pyridin-2-yl-thiazol-4-yl)-thieno[2,3-b]pyridine,
3-(4-chloro-phenyl)-2-(2-thiophen-2-yl-oxazol-4-yl)-thieno[2,3-b]pyridine,
{4-[3-(4-chloro-phenyl)-thieno[2,3-b]pyridin-2-yl]-thiazol-2-yl}-ethyl-amine,
N-{4-[3-(4-chloro-phenyl)-thieno[2,3-b]pyridin-2-yl]-thiazol-2-yl}-acetamide,
3-(4-chloro-phenyl)-6-methyl-2-(5-methyl-isoxazol-3-yl)-thieno[2,3-b]pyridine,
3-(4-chloro-phenyl)-2-(5-methyl-isoxazol-3-yl)-thieno[2,3-b]pyridine,
5-[3-(4-chloro-phenyl)-thieno[2,3-b]pyridin-2-yl]-2-methyl-furan-3-carboxylic acid methyl ester,
3-(4-chloro-phenyl)-2-(3-ethyl-[1,2,4]oxadiazol-5-yl)-thieno[2,3-b]pyridine,
3-(4-chloro-phenyl)-2-[3-(4-fluoro-phenyl)-[1,2,4]oxadiazol-5-yl]-thieno[2,3-b]pyridine, and
3-(4-fluoro-phenyl)-2-[5-(4-fluoro-phenyl)-4,5-dihydro-isooxazol-3-yl]-thieno[2,3-b]pyridine.

21. A process for preparing a compound of formula (I): 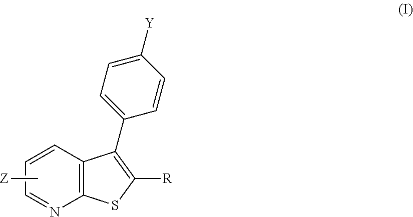
wherein Y is selected from hydrogen, alkyl, halogen, and alkoxy, when
Z is hydrogen or alkyl and
R is an optionally substituted heteroaryl, and Y is selected from hydrogen, methyl, fluoro, chloro, bromo, and methoxy, when Z is hydrogen or methyl, and R is a monocyclic or bicyclic heteroaryl ring containing 1-4 heteroatom(s) selected from O, N and S, which is optionally substituted with one or more alkyl, alkoxy, halogen, methoxycarbonyl, amino, alkylamino, acylamino, optionally substituted phenyl or a monocyclic or bicyclic heteroaryl ring containing 1-4 heteroatom(s) selected from O, N and S, comprising:
reacting a compound of formula (IV): 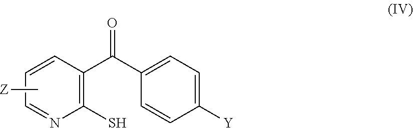
wherein the meaning of Z and Y is as described above for the compound of formula (I),
with a compound of formula (VI):
HlgCH₂R (VI)
wherein Hlg is chloro or bromo, R is as defined above for the compound of formula (I) in the presence of a base in a solvent under reflex or in a microwave reactor;
and, optionally thereafter forming salts, hydrates, or solvates of compounds of formula (I).

22. A pharmaceutical formulation comprising a therapeutically effective amount of a compound of formula (I): 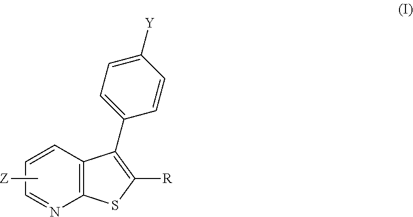
wherein
Y is selected from hydrogen, alkyl, halogen, and alkoxy, when Z is hydrogen or alkyl and R is an optionally substituted heteroaryl, and
Y is selected from hydrogen, methyl, fluoro, chloro, bromo, and methoxy, when Z is hydrogen or methyl, and R is a monocyclic or bicyclic heteroaryl ring containing 1-4 heteroatom(s) selected from O, N and S, which is optionally substituted with one or more alkyl, alkoxy, halogen, methoxycarbonyl, amino, alkylamino, acylamino, optionally substituted phenyl or a monocyclic or bicyclic heteroaryl ring containing 1-4 heteroatom(s) selected from O, N and S; and,
at least one physiologically acceptable diluent, excipient or inert carrier.

23. A method for treating mGluR1 or mGluR5 receptor-mediated disorders, comprising administering a formulation according to claim 22 to a mammal in need of treatment for mGluR1 or mGluR5 receptor-mediated disorders.

24. The method of claim 23, wherein said mGluR1 or mGluR5 receptor-mediated disorders are psychiatric disorders.

25. The method of claim 23, wherein said mGluR1 or mGluR5 receptor-mediated disorders are psychiatric disorders.

26. The method of claim 23, wherein said mGluR1 or mGluR5 receptor-mediated disorders are neurological disorders.

27. The method of claim 23, wherein said mGluR1 or mGluR5 receptor-mediated disorders are neurological disorders.

28. The method of claim 23, wherein said mGluR1 or mGluR5 receptor-mediated disorders are chronic and acute pain.

29. The method of claim 23, wherein said mGluR1 or mGluR5 receptor-mediated disorders are chronic and acute pain.

30. The method of claim 23, wherein said mGluR1 or mGluR5 receptor-mediated disorders are neuromuscular dysfunctions of the lower urinary tract.

31. The method of claim 23, wherein said mGluR1 or mGluR5 receptor-mediated disorders are neuromuscular dysfunctions of the lower urinary tract.

32. The method of claim 23, wherein said mammal is a human.

33. A method for treating mGluR1 or mGluR5 receptor-mediated disorders, comprising administering a therapeutically effective amount of a compound according to claim 18 to a mammal in need of treatment for mGluR1 or mGluR5 receptor-mediated disorders.

34. A method for treating mGluR1 or mGluR5 receptor-mediated disorders, comprising administering a therapeutically effective amount of a compound according to claim 19 to a mammal in need of treatment for mGluR1 or mGluR5 receptor-mediated disorders.
